(19) United States
(12) Patent Application Publication
(10) Pub. No.: US 2008/0058904 A1
(43) Pub. Date: Mar. 6, 2008

(54) Electromagnetic device and method

(75) Inventors: W. Daniel Hillis, Encino, CA (US); Leroy E. Hood, Seattle, WA (US); Roderick A. Hyde, Redmond, WA (US); Eric C. Leuthardt, St. Louis, MO (US); Nathan P. Myhrvold, Medina, WA (US); Clarence T. Tegreene, Bellevue, WA (US); Lowell L. Wood, Bellevue, WA (US)

Correspondence Address: Searete LLC, Suite 110, 1756 - 114th Ave. S.E., Bellevue, WA, 98004, US

(73) Assignee: Searete LLC, a limited liability corporation of the State of Delaware

(21) Appl. No.: 11/731,788

(22) Filed: Mar. 30, 2007

Related U.S. Application Data
(63) Continuation-in-part of application No. 11/515,412, filed on Aug. 31, 2006.

(51) Int. Cl. A61N 5/06

(52) U.S. Cl. 607/88; 359/485; 359/641; 359/710; 359/712; 359/728; 359/869

(57) ABSTRACT

Embodiments include an apparatus, a medical device, a method and a system. The medical device includes an ellipsoidally shaped reflector having a first focus and a second focus. The ellipsoidally shaped reflector also provides a translational coupling of electromagnetic energy from the first focus to the second focus. The medical device also includes a controllable electromagnetic energy source aligned to emit a non-biologically emitted electromagnetic energy in a proximity to the first focus.

### CROSS-REFERENCE TO RELATED APPLICATIONS

The present application is related to and claims the benefit of the earliest available effective filing date(s) from the following listed application(s) (the “Related Applications”) (e.g., claims earliest available priority dates for other than provisional patent applications or claims benefits under 35 USC § 119(e) for provisional patent applications, for any and all parent, grandparent, great-grandparent, etc. applications of the Related Application(s)).

### RELATED APPLICATIONS

For purposes of the USPTO extra-statutory requirements, the present application constitutes a continuation-in-part of U.S. patent application Ser. No. 11/515,412, entitled ELECTROMAGNETIC DEVICE AND METHOD, naming W. Daniel Hillis, Leroy E. Hood, Roderick A. Hyde, Eric C. Leuthardt, Nathan P. Myhrvold, Clarence T. Tegreene and Lowell L. Wood, Jr. as inventors, filed 31 Aug. 2006, which is currently co-pending, or is an application of which a currently co-pending application is entitled to the benefit of the filing date.

The United States Patent Office (USPTO) has published a notice to the effect that the USPTO's computer programs require that patent applicants reference both a serial number and indicate whether an application is a continuation or continuation-in-part. Stephen G. Kunin, Benefit of Prior-Filed Application, USPTO Official Gazette Mar. 18, 2003, available at http://www.uspto.gov/web/offices/com/sol/og/2003/week11/patbene.htm. The present Applicant Entity (hereinafter “Applicant”) has provided above a specific reference to the application(s) from which priority is being claimed as recited by statute. Applicant understands that the statute is unambiguous in its specific reference language and does not require either a serial number or any characterization, such as “continuation” or “continuation-in-part,” for claiming priority to U.S. patent applications. Notwithstanding the foregoing, Applicant understands that the USPTO's computer programs have certain data entry requirements, and hence Applicant is designating the present application as a continuation-in-part of its parent applications as set forth above, but expressly points out that such designations are not to be construed in any way as any type of commentary and/or admission as to whether or not the present application contains any new matter in addition to the matter of its parent application(s).

All subject matter of the Related Applications and of any and all parent, grandparent, great-grandparent, etc. applications of the Related Applications is incorporated herein by reference to the extent such subject matter is not inconsistent herewith.

### TECHNICAL FIELD

An aspect of the present application relates, in general, to devices, methods and/or systems for treatment and/or management of disease, disorders, or conditions.

### SUMMARY

An embodiment provides a medical device. In one aspect, the medical device includes an ellipsoidally shaped reflector having a first focus and a second focus. The ellipsoidally shaped reflector provides a translational coupling of electromagnetic energy from said first focus to said second focus. The medical device also includes a controllable electromagnetic energy source aligned to emit a non-biologically emitted electromagnetic energy in a proximity to the first focus.

In an alternative embodiment, the ellipsoidally shaped reflector includes an opening configured to allow positioning of a portion of a body proximate with said second focus. A further embodiment of the ellipsoidally shaped reflector includes a conductor. In another embodiment, the conductor includes at least one of an aluminum, a tin, a stainless steel, a silver, a gold, a copper, an iron, a carbon, an iridium, an indium, a lead, a magnesium, a nickel, a nichrome, a palladium, a rhodium, a silver, a tantalum, a titanium, a tungsten, a zinc, a platinum and/or a zirconium. In yet another embodiment, the ellipsoidally shaped reflector includes a dielectric material. In a further embodiment, the dielectric material includes a rubber, a plastic, a porcelain, a ceramic, a mica, a glass, a metal oxide, a perfect vacuum, a dry air, a pure dry gas, a helium and/or a nitrogen.

In an alternative embodiment, the non-biologically emitted electromagnetic energy includes at least one of a visible light, a laser energy, an ultraviolet energy, an infrared energy, an X-ray or a microwave. In an embodiment, the controllable electromagnetic energy source includes a computer configured to control delivery of the non-biological electromagnetic energy. In a further embodiment, the controllable electromagnetic energy source includes at least one of a plano-convex lens, a meniscus lens, a cylindrical lens, a parabolic lens, an acrylic lens, a glass lens, a quartz lens, a Fresnel lens, a Pendry lens, a band pass filter, a polarizer, a dichroic material, a monochromator and/or a collimator. In yet another embodiment, the controllable electromagnetic energy source regulates at least a characteristic of the non-biologically emitted electromagnetic energy. In a further embodiment, the at least a characteristic of the non-biologically emitted electromagnetic energy includes at least one of a wavelength, a frequency, an amplitude, a phase, a polarization and/or a bandwidth. In addition to the foregoing, other embodiments of the medical device described in the claims, drawings, and text form a part of the patent application.

An embodiment provides a method of treating a portion of a living body. The method includes emitting a selected dose corresponding to a predicted therapeutic level of electromagnetic energy in a proximity to a first focus of an ellipsoid. The method also includes translating the selected dose corresponding to a predicted therapeutic level of electromagnetic energy to a second focus of the ellipsoid. The method further includes activating a biological tissue in a proximity to the second focus with the selected dose corresponding to a predicted therapeutic level of electromagnetic energy.

In an embodiment of the foregoing method, the emitting includes emitting electromagnetic energy having at least one wavelength between 1 nm and 700 nm; 2.0 μm and 10 μm; and/or 1 cm and 100 cm. In another illustrative embodiment, the emitting a selected dose of electromagnetic energy includes electromagnetic energy having at least one wavelength between 700 and 2000 nm. In a further illustrative embodiment, the emitting a selected dose of electromagnetic energy includes electromagnetic energy having at least one wavelength between 10 μm and 1 cm. In a further embodiment, the emitting includes electromagnetic energy having at least one of an amplitude variation, a phase variation and/or a variable polarization parameter. In yet another embodiment of the foregoing method, the emitting electromagnetic energy as a series of one or more pulses, each of the pulses having at least a pulse duration between a picosecond and a second.

In a further embodiment of the method, the translating includes the second focus having a volume between 1000 μm³ and 1000 cm³ in a proximity to a biological tissue.

In another embodiment of the foregoing method, the activating a biological tissue includes selectively energizing the first portion of the biological tissue differentially relative to a second portion of the biological tissue. In yet another embodiment, the activating includes coverage of 0.1% to 100% of the biological tissue with the second focus. In an embodiment of the method includes activating the biological tissue using electromagnetic energy having a level between 1 to 100,000 milli Joules per gram of biological tissue at the second focus. In a further embodiment, the activating includes making the second focus at least substantially coincidental with a first portion of the biological tissue and then making the second focus at least substantially coincidental with a second portion of the biological tissue. In addition to the foregoing, other embodiments of the method described in the claims, drawings, and text form a part of the patent application.

A medical device for treating a portion of a living body is provided. The medical device includes a means for emitting a selected dose corresponding to a predicted therapeutic level of electromagnetic energy in a proximity to the first focus of the ellipsoid. The medical device also provides for a means for translating the selected dose corresponding to a predicted therapeutic level of electromagnetic energy to the second focus of the ellipsoid. The medical device provides a means for activating a biological tissue in a proximity to the second focus with the selected dose corresponding to a predicted therapeutic level of electromagnetic energy.

An embodiment provides a system of treating a portion of a living body. The system includes an ellipsoidally shaped reflector having a first focus and a second focus, and shaped to provide a translational coupling of electromagnetic energy from the first focus to the second focus. The system also includes a controllable electromagnetic energy source aligned to emit a non-biologically emitted electromagnetic energy in a proximity to the first focus. The system further includes an electromagnetic energy source controller coupled to the energy source and having a regulator. The regulator includes electrical circuitry configured to govern at least one of a wavelength, an amplitude, a polarization state, a bandwidth, a collimation filter, a phase shift, a pulse, a frequency and/or a focus. In addition to the foregoing, other embodiments of the system described in the claims, drawings, and text form a part of the patent application.

The foregoing summary is illustrative only and is not intended to be in any way limiting. In addition to the illustrative aspects, embodiments, and features described above, further aspects, embodiments, and features will become apparent by reference to the drawings and the following detailed description.

### BRIEF DESCRIPTION OF THE FIGURES

FIG. 1 illustrates an exemplary general-purpose medical device in which embodiments may be implemented;

FIG. 2 is a perspective view of an embodiment of an ellipsoidally shaped reflector showing an embodiment of a conductor and dielectric material;

FIG. 3 illustrates an exemplary operational flow in which embodiments may be implemented;

FIG. 4 illustrates an alternative embodiment of the exemplary operational flow of FIG. 3;

FIG. 5 illustrates an alternative embodiment of the exemplary operational flow of FIG. 3;

FIG. 6 illustrates an alternative embodiment of the exemplary operational flow of FIG. 3;

FIG. 7 schematically illustrates a simplified medical device in which an embodiment of the exemplary operation flow of FIG. 3 may be implemented;

FIG. 8 schematically illustrates a simplified medical device in which an embodiment of the exemplary operation flow of FIG. 3 may be implemented;

FIG. 9 schematically illustrates a simplified medical device in which an embodiment of the exemplary operation flow of FIG. 3 may be implemented;

FIG. 10 schematically illustrates a simplified medical device in which an embodiment of the exemplary operation flow of FIG. 3 may be implemented;

FIG. 11 schematically illustrates a simplified medical device in which an embodiment of the exemplary operation flow of FIG. 3 may be implemented;

FIG. 12 illustrates an exemplary medical device that may be used to implement embodiments; and

FIG. 13 illustrates an exemplary system that may be used to implement embodiments.

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented here.

The following disclosure is drawn to a medical device comprising an ellipsoidally shaped reflector having a first focus and a second focus, and providing a translational coupling of electromagnetic energy from the first focus to the second focus. In an aspect, the disclosure is drawn to a medical device comprising a half ellipsoid configured to, and/or structured to at least partially or completely be coupled to a controllable electromagnetic energy source aligned to emit a non-biologically emitted electromagnetic energy in a proximity to the first focus and includes an opening configured to at least partially or completely allow the positioning of at least a portion of a living body or a biological tissue in proximity to the second focus.

In some embodiments, the medical device is a structure comprising a fully or partially enclosed ellipsoid configured to, and/or structured to at least partially or completely be aligned to a controllable electromagnetic energy source aligned to emit a non-biologically emitted electromagnetic energy in a proximity to the first focus and includes an opening configured to at least partially or completely allow the positioning of a portion of an animal body proximate with the second focus.

In other embodiments, the medical device is a structure enclosing a substructure comprising a fully or partially enclosed ellipsoid configured to, and/or structured to at least partially or completely be aligned to a controllable electromagnetic energy source aligned to emit a non-biologically emitted electromagnetic energy in a proximity to the first focus and includes an opening configured to at least partially or completely allow the positioning of a portion of an animal body proximate with the second focus.

In an embodiment, the term “full ellipsoid” describes a structure that substantially encloses an ellipsoid or an ellipsoidally shaped structure having one or more openings.

In an embodiment, the term “partial ellipsoid” in reference to a structure or substructure includes a structure or substructure comprising a lengthwise cross-section along the major axis of an ellipsoidally shaped structure or substructure.

In an embodiment, the term “living body” refers to a human or any animal including domestic, marine, research, zoo, farm animals, fowl and sports animals, or pet animals, such as dogs, cats, cattle, horses, sheep, pigs, goats, rabbits, chicken, birds, fish, amphibian and reptile.

As used herein, the term “biological tissue” includes any portion of a living body or anatomy or morphology or a part of a physiology of a living body including intact or fragmented or sheared or isolated living or dead biological tissue or cells in culture or isolated cells in vitro or in vivo or ex vivo or individual colonies of microbial/eukaryotic cells or a single isolated cell. The biological tissue may include microbes, viruses, tissue isolates from living or non-living animals and/or plants.

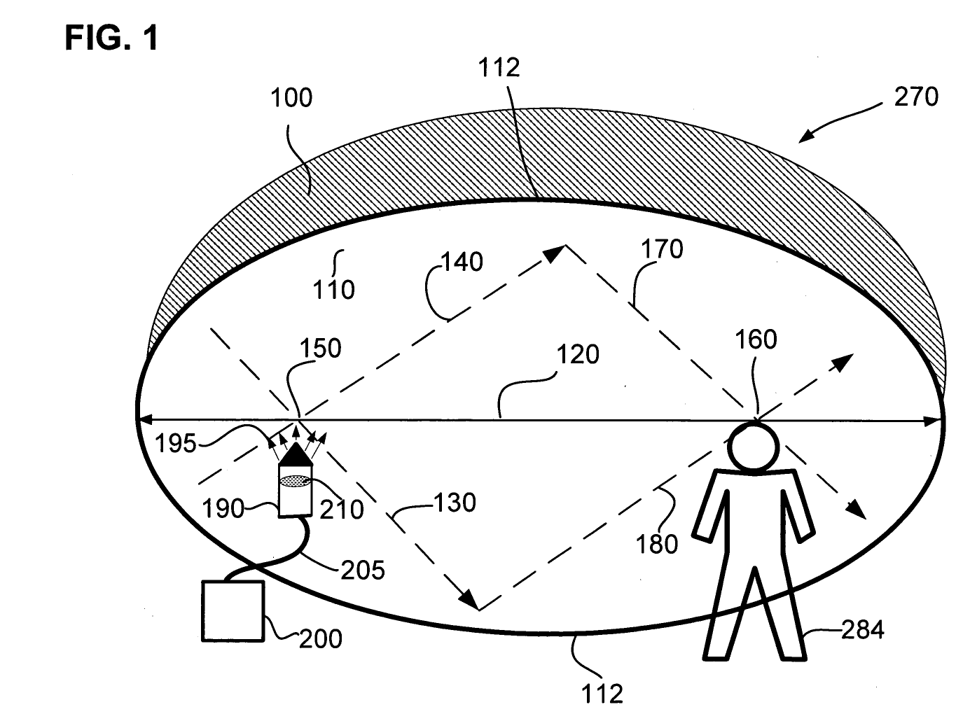

FIG. 1 illustrates an exemplary general-purpose medical device 270 in which embodiments may be implemented. The medical device comprises an ellipsoidally shaped reflector 100 having a first focus 150 and a second focus 160, and providing a translational coupling 120 of non-biological electromagnetic energy 195 from the first focus to the second focus. The medical device also includes a controllable electromagnetic energy source 200 aligned to emit a non-biologically emitted electromagnetic energy 195 in a proximity to the first focus. In another embodiment, incident electromagnetic rays 130 and 140 pass through the first focus and reflect off the reflective surface 10 and form reflected rays 170 and 180, respectively, and converge at the second focus.

In a further embodiment, the ellipsoidally shaped reflector 100 includes an opening 112 configured to allow positioning of a biological tissue of a living body 284 proximate with the second focus 160.

In another embodiment, at least of a portion of the reflector includes at least one of a metal, a dielectric, a liquid, a multilayer, a crystal, and/or a Bragg reflector.

In another embodiment, the non-biologically emitted electromagnetic energy 195 includes at least one of a visible light, a laser energy, an ultraviolet energy, an infrared energy, an X-ray and/or a microwave. In yet another embodiment, the electromagnetic energy emitter 190 is coupled 205 to an electromagnetic energy source 200 configured for controlled delivery of the non-biological electromagnetic energy 195. In another embodiment, the controllable electromagnetic energy source includes at least one 210 of a plano-convex lens, a meniscus lens, a cylindrical lens, a parabolic lens, an acrylic lens, a glass lens, a quartz lens, a Fresnel lens, a Pendry lens, a band pass filter, a polarizer, a dichroic material, a monochromator and/or a collimator. In another embodiment, the controllable electromagnetic energy source regulates at least a characteristic of the non-biologically emitted electromagnetic energy. In a further embodiment, the at least a characteristic of the non-biologically emitted electromagnetic energy includes at least one of a wavelength, a frequency, an amplitude, a phase, a polarization and/or a bandwidth.

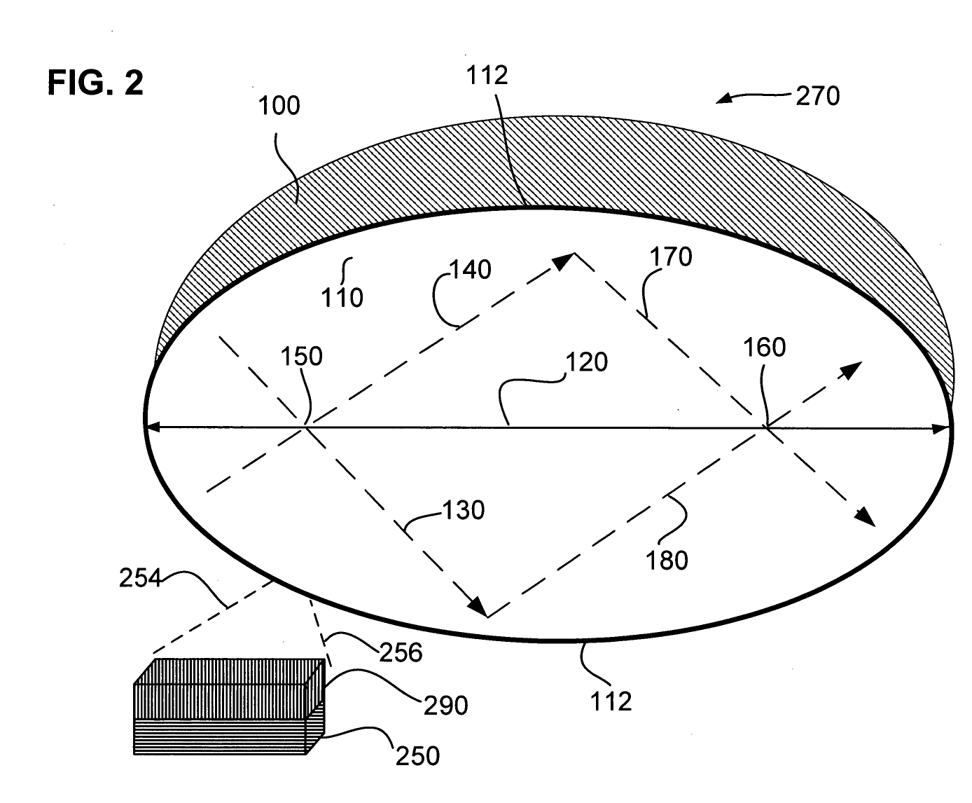

FIG. 2 shows an embodiment of the medical device 270 that includes a conductor 250 and a dielectric material 290. In an embodiment, an exploded view 254-256 depicts the conductor and the dielectric material. In yet another embodiment, the conductor includes at least one of an aluminum, a tin, a stainless steel, a silver, a gold, a copper, an iron, a carbon, an iridium, an indium, a lead, a magnesium, a nickel, a nichrome, a palladium, a rhodium, a silver, a tantalum, a titanium, a tungsten, a zinc, a platinum, and/or a zirconium. In other embodiments, the dielectric material includes a rubber, a plastic, a porcelain, a ceramic, a mica, a glass, a plastics, a metal oxide, a perfect vacuum, a dry air, a pure dry gas such as helium and/or nitrogen.

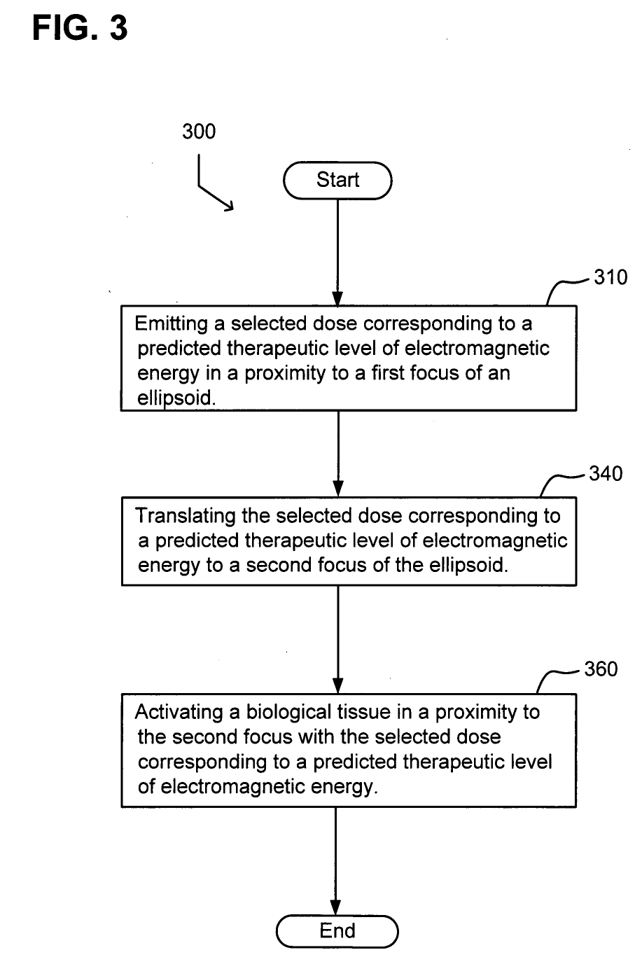

FIG. 3 illustrates an exemplary operational flow 300 in which embodiments may be implemented. After a start operation, the operational flow moves to a radiating operation 310. The radiating operation emits a selected dose corresponding to a predicted therapeutic level of electromagnetic energy in a proximity to a first focus of an ellipsoid. A transposing operation 340 translates a selected dose corresponding to a predicted therapeutic level of electromagnetic energy to a second focus of the ellipsoid. An irradiation operation 360 activates a biological tissue in a proximity to the second focus with the selected dose corresponding to a predicted therapeutic level of electromagnetic energy. The operational flow moves to a stop operation.

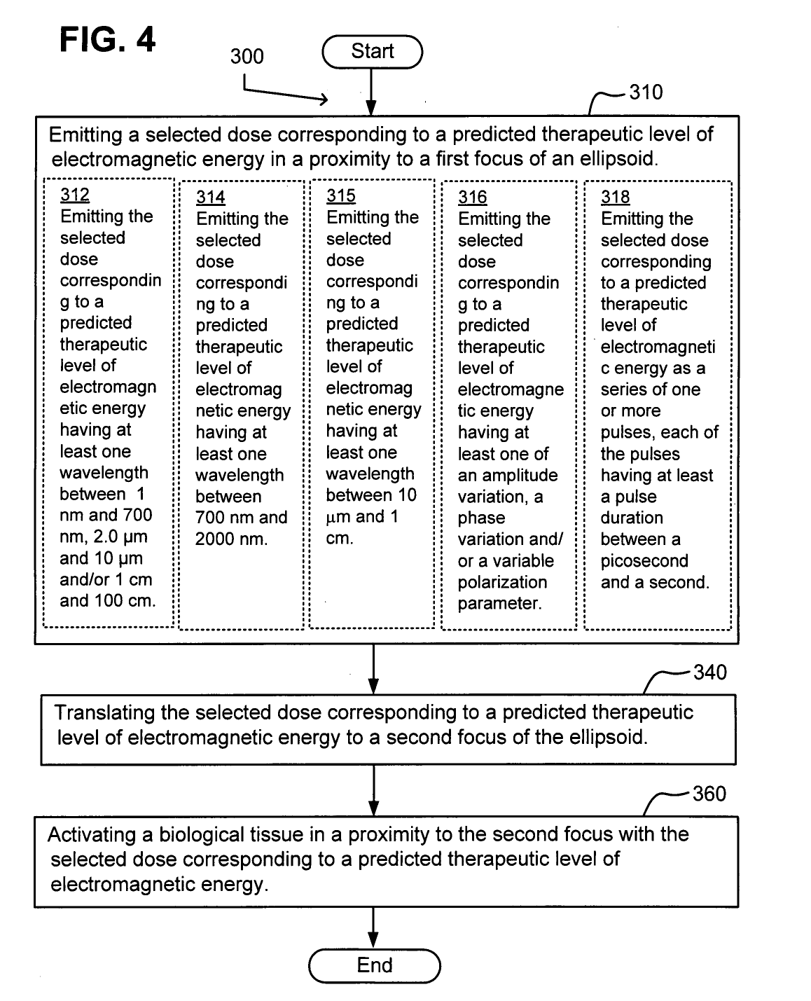

FIG. 4 illustrates an alternative embodiment of the exemplary operational flow 300 of FIG. 3. The radiating operation 310 may include at least one additional operation. The at least one additional operation may include an operation 312, an operation 314, an operation 316 and/or an operation 318. The operation 312 emits the selected dose corresponding to a predicted therapeutic level of electromagnetic energy having at least one wavelength between 1 nm and 700 nm, 2.0 μm and 10 μm and/or 1 cm and 100 cm. The operation 314 emits the selected dose corresponding to a predicted therapeutic level of electromagnetic energy having at least one wavelength between 700 and 2000 nm. The operation 315 emits the selected dose corresponding to a predicted therapeutic level of electromagnetic energy having at least one wavelength between 10 μm and 1 cm. The operation 316 emits the selected dose corresponding to a predicted therapeutic level of electromagnetic energy having at least one of an amplitude variation, a phase variation and/or a variable polarization parameter. The operation 318 emits the selected dose corresponding to a predicted therapeutic level of electromagnetic energy as a series of one or more pulses, each of the pulses having at least a pulse duration between a picosecond and a second.

In another embodiment, the wavelength of a selected dose corresponding to a predicted therapeutic level of electromagnetic energy includes at least the following wavelength ranges: from 1 nm to 10 nm; from 10 nm to 100 nm; from 100 nm to 700 nm; from 700 nm to 800 nm; from 800 nm to 900 nm; from 900 nm to 1000 nm; from 1000 nm to 1300 nm; from 1300 nm to 1700 nm; from 1700 nm to 2000 nm; from 2 μm to 3 μm; 3 μm to 5 μm; from 5 μm to 10 μm; 10 μm to 20 μm; from 20 μm to 30 μm; from 30 μm to 40 μm; from 40 μm to 50 μm; from 50 μm to 100 μm and from 100 μm to 1000 μm.

In other embodiments, the wavelength of a selected dose corresponding to a predicted therapeutic level of electromagnetic energy includes at least the following wavelength ranges: 0.1 cm to 0.5 cm; from 0.5 cm to 1 cm; from 1 cm to 5 cm; from 5 cm to 10 cm; from 10 cm to 20 cm; from 20 cm to 30 cm; from 30 cm to 40; from 50 cm to 60 cm and from 60 to 100 cm.

One of skill in the art will appreciate that in some embodiments, ranges of wavelength, frequency, amplitude, phase, polarization and/or a bandwidth and/or combinations thereof for selected doses corresponding to predicted therapeutic levels of electromagnetic energy may be utilized for different biological tissues and/or for different types of conductors in the ellipsoidal shaped reflector.

In some embodiments, the phrase “a selected dose corresponding to a predicted therapeutic level” of non-biological electromagnetic energy includes an energy level that is intended for delivery at a portion of a biological tissue and/or cell(s) to achieve inter alia a palliative and/or curative and/or therapeutic treatment and/or maintain/achieve a desired result for an animal patient or human patient or a research subject.

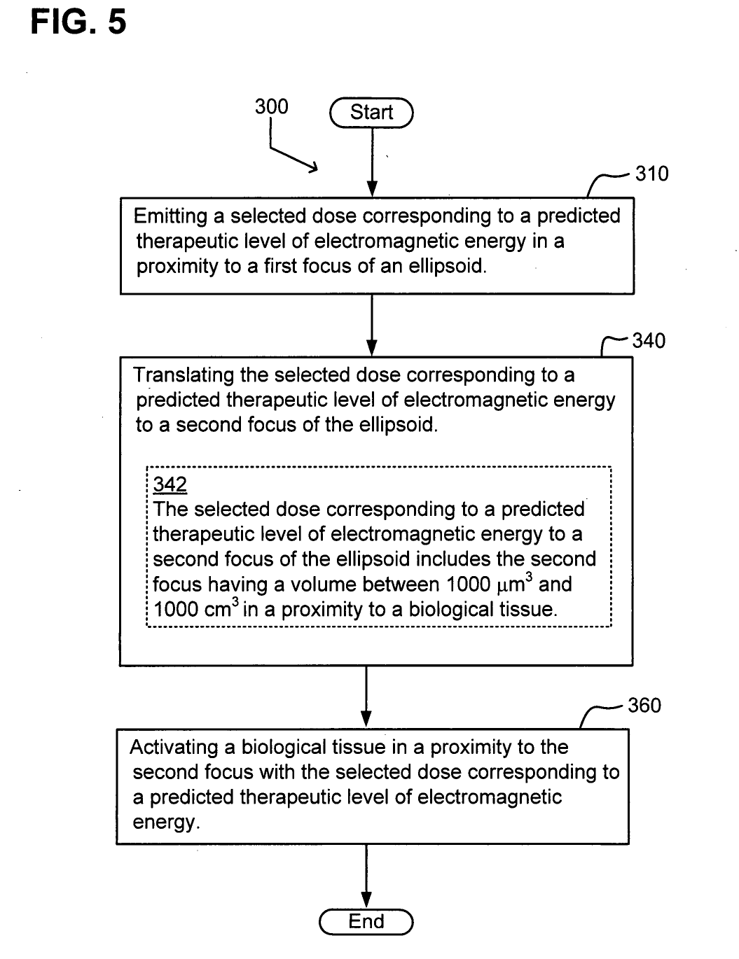

FIG. 5 illustrates an alternative embodiment of the exemplary operational flow 300 of FIG. 3. The transposing operation 340 may include at least one additional operation. The at least one additional operation may include an operation 342. The operation 342 translates the selected dose corresponding to a predicted therapeutic level of electromagnetic energy to a second focus of the ellipsoid, which includes the second focus having a volume between 1000 μm³ and 1000 cm³ in a proximity to a biological tissue.

In other embodiments, the second focus has a volume that includes at least the following ranges: from 1000 μm³ to 10,000 μm³; from 0.01 mm³ to 0.1 mm³; from 0.1 mm³ to 0.5 mm³; from 0.5 mm³ to 0.7 mm³; from 0.7 mm³ to 0.9 mm³; from 0.9 mm³ to 1.1 mm³; from 1.1 mm³ to 1.3 mm³; from 1.3 mm³ to 1.5 mm³; 1.5 mm³ to 2.0 mm³; from 2 mm³ to 5 cm³; from 5 cm³ to 10 cm³; from 10 cm³ to 100 cm³ and/or from 100 cm³ to 1000 cm³.

One of skill in the art will appreciate that in some embodiments, a single second focal volume and/or a single power and/or a multiplicity of ranges of focal volumes and/or power and/or combinations thereof may be utilized for different biological tissues and/or for different types of conductors in the ellipsoidal shaped reflector.

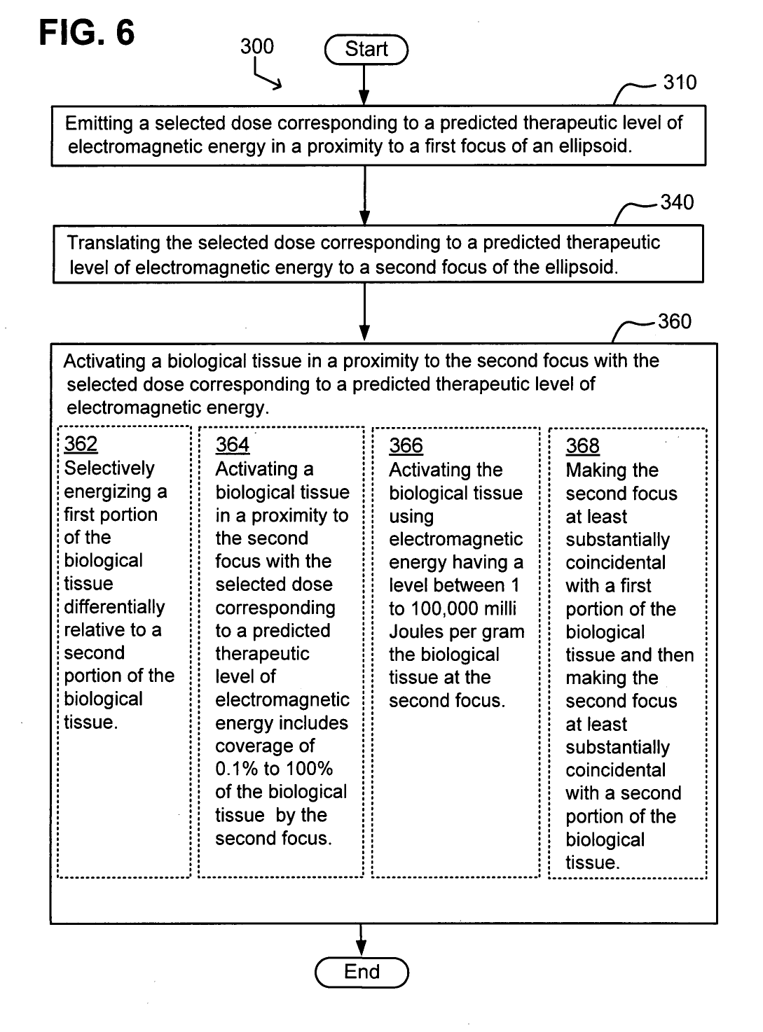

FIG. 6 illustrates an alternative embodiment of the exemplary operational flow 300 of FIG. 3. The irradiation operation 360 may include at least one additional operation. The at least one additional operation may include an operation 362, an operation 364, an operation 366 and/or an operation 368. The operation 362 selectively energizes a first portion of the biological tissue differentially relative to a second portion of the biological tissue. The activating operation 364 achieves a coverage of 0.1% to 100% of the biological tissue with the second focus. The activation operation 366 activates the biological tissue using electromagnetic energy having a level between 1 to 100,000 milli Joules per gram of biological tissue at the second focus. The activation operation 368 makes the second focus at least substantially coincidental with a first portion of the biological tissue and then making the second focus at least substantially coincidental with a second portion of the biological tissue.

In some embodiments the coverage of second focus with the selected dose corresponding to a predicted therapeutic level of electromagnetic energy includes coverage of approximately from 0.1% to 1%; from 1% to 10%; from 10% to 20%; from 20% to 30%; from 30% to 40%; from 40% to 50%; from 50% to 60%; from 60% to 70%; from 70% to 80%; from 80% to 90%; and from 90% to 100%. One of skill in the art will recognize that the extent of coverage by second focus with the selected dose corresponding to a predicted therapeutic level of electromagnetic energy depends on the size, depth and shape of the target biological tissue. One of skill in the art will also recognize that the extent of coverage by second focus with the selected dose corresponding to a predicted therapeutic level of electromagnetic energy is related to the size of the focal area of the second focus vis-à-vis the size of the target biological tissue. For instance, if the size of the target biological tissue is larger than the second focal area then the effective coverage by second focus with the selected dose corresponding to a predicted therapeutic level of electromagnetic energy will be less than 100% of the target biological tissue. One of skill in the art will recognize that in this case a plurality of activation regimes at second focus with the selected dose corresponding to a predicted therapeutic level of electromagnetic energy will be necessary to achieve 100% coverage of the target biological tissue. In this case, activation regimes can be adjusted to cover the target biological tissue in increments of less than 100% coverage at one time and repeating activation regimes at second focus multiple times until the desired level of coverage is achieved.

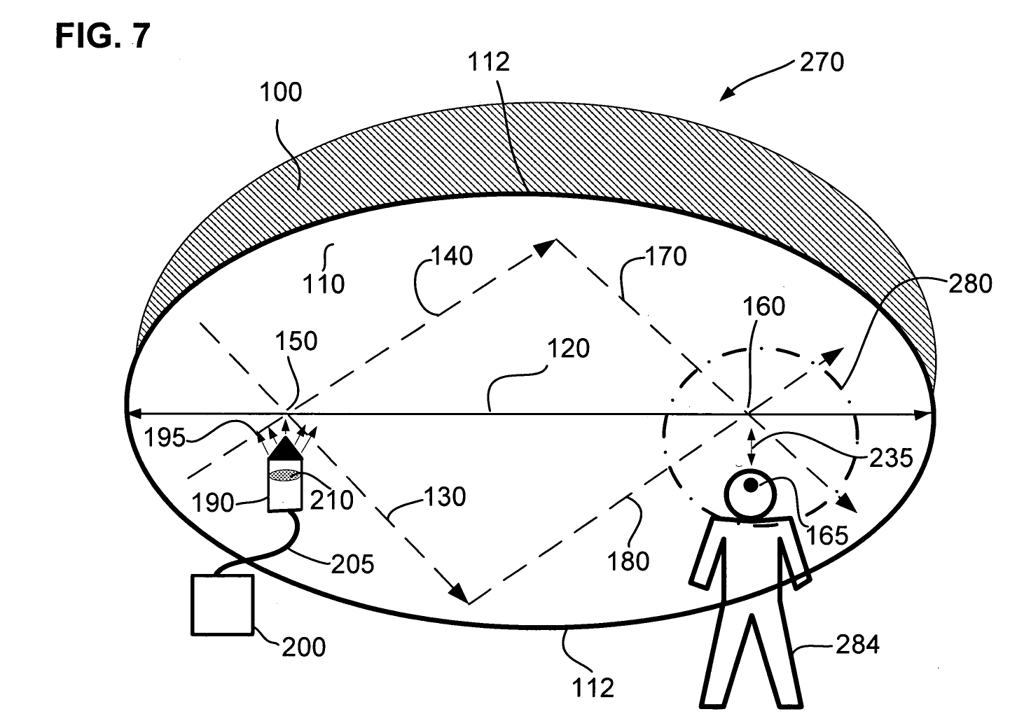

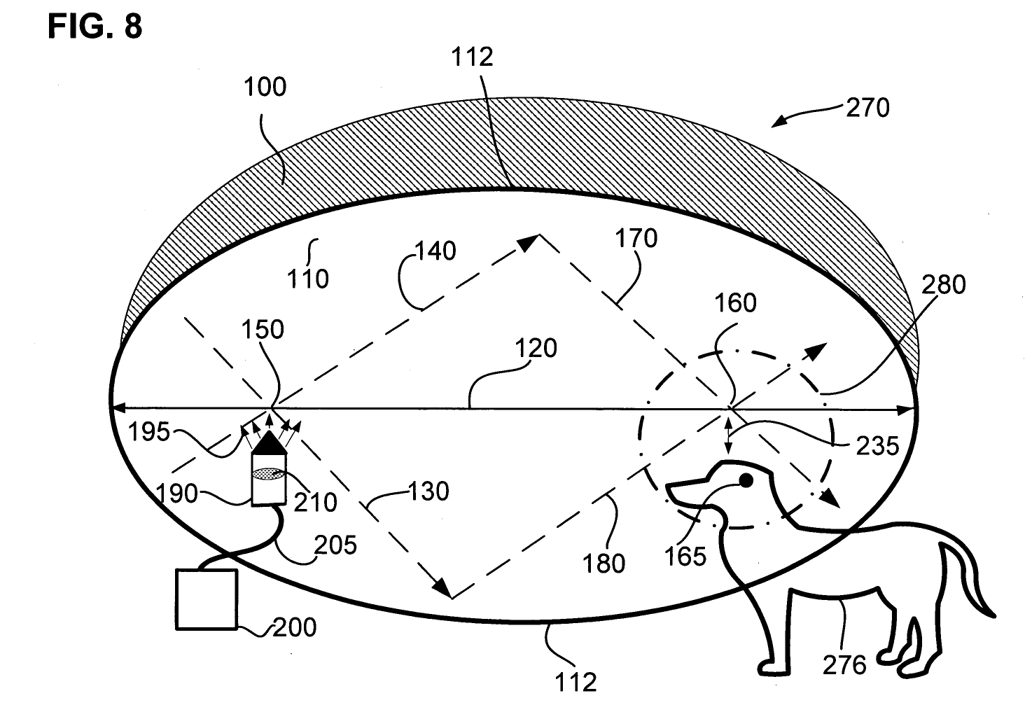

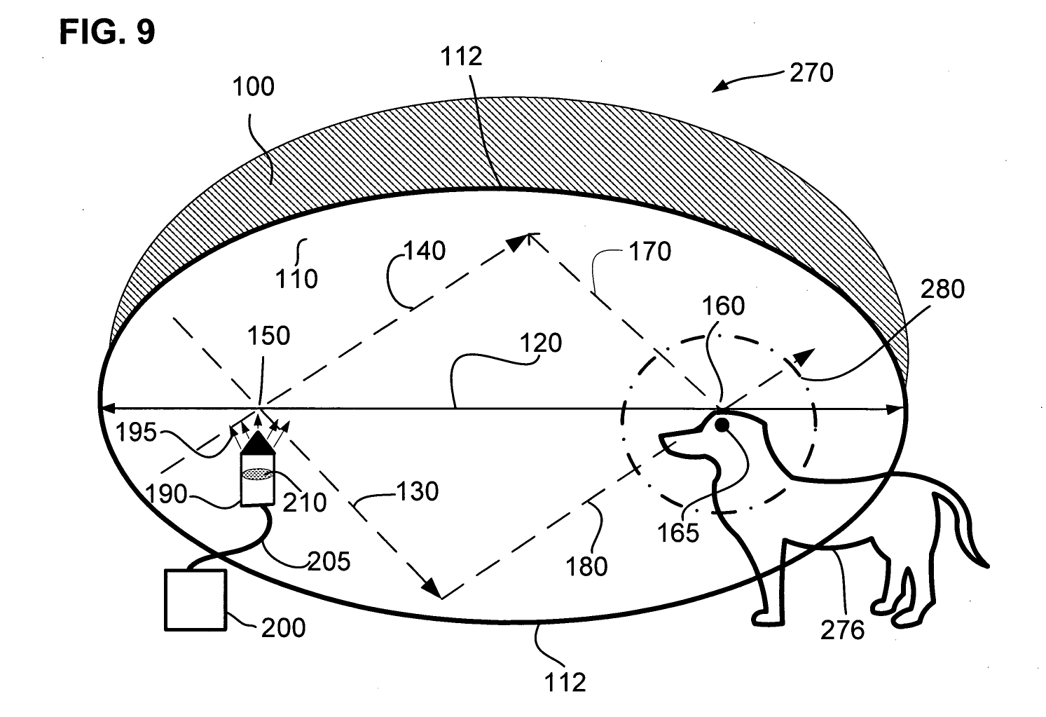

FIGS. 7 through 11 schematically illustrate the medical device 270 in which an embodiment of the exemplary operation flow 300 of FIG. 3 may be implemented. FIGS. 7-9 depict alternative embodiments, which may be implemented in a zone of activation 280. Biological tissue 165 from a human body 284 may be activated in the zone of activation. The medical device 270 provides irradiation either in a proximity 235 (FIGS. 7 and 8) to the second focus 160 or irradiation in substantial coincidence (FIG. 9) with the second focus. One of skill in the art will appreciate that the proximity of the second focus may be achieved by either moving the ellipsoid reflector or moving the living body and/or moving both depending on a required therapeutic dosage and/or dimensions of the living body.

FIGS. 8 and 9 depict a dog 276 as the subject/patient for treatment. In FIG. 8 the biological tissue 165 is in a proximity 235 to the second focus 160 whereas in FIG. 9 the biological tissue is in substantial coincidence with the second focus. In principle, any animal may be substituted for the dog in FIGS. 8 and 9.

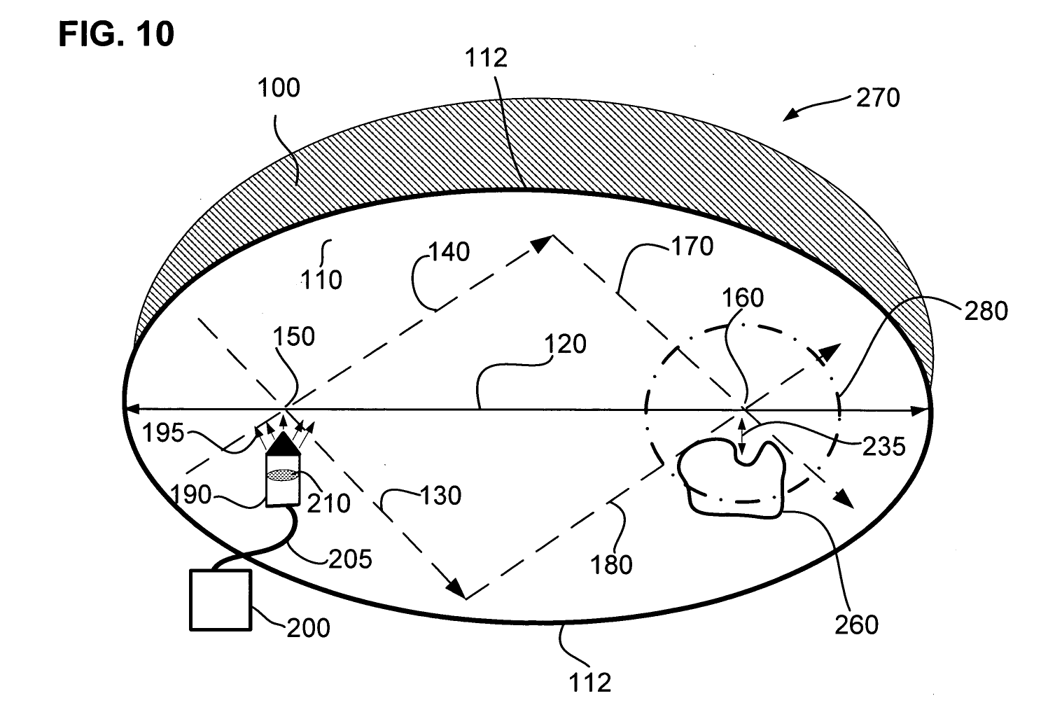

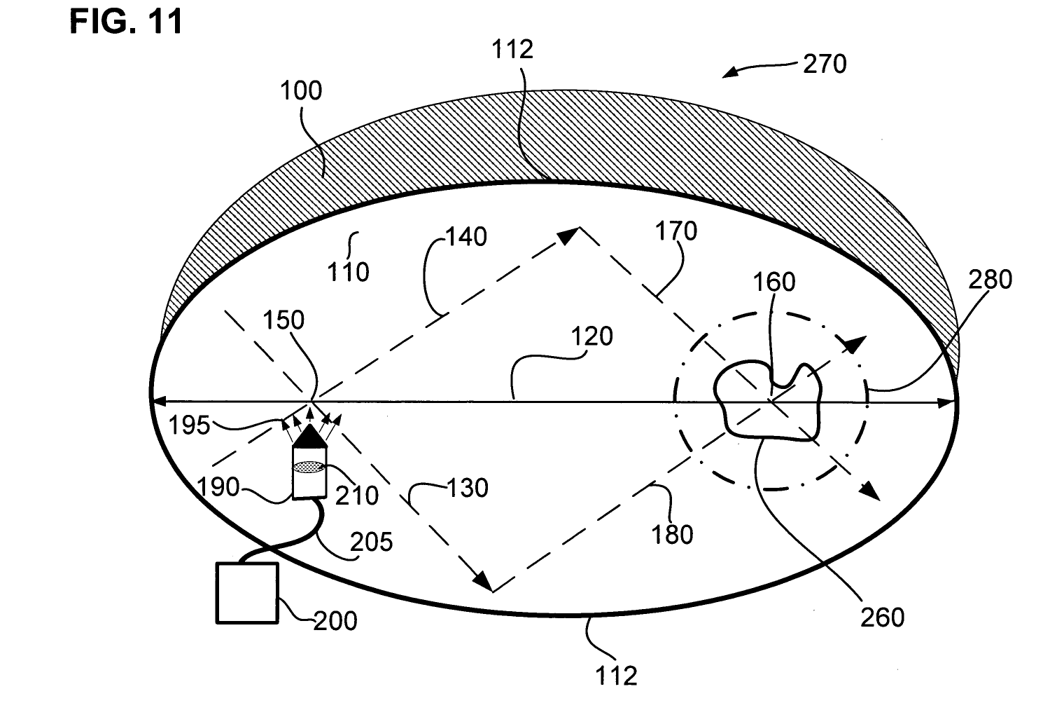

FIGS. 10 and 11 depict further embodiments, which may be implemented in the medical device 270. Herein, isolated biological tissue 260 is treated in the zone of activation 280 either in a proximity (FIG. 10) to the second focus or treated in substantial coincidence (FIG. 11) with the second focus.

One of skill in the art will appreciate that differentially irradiating different portions of the biological tissue may include changing wavelength, amplitude, phase, polarization, power, focal volume, focal depth and/or focal area of the second focus.

Some alternative embodiments may be implemented in the medical device 270 for activation of the biological tissue 165 include administration of a plurality of temporally spaced irradiations of the biological tissue. For example, a portion of the biological tissue may be activated first for certain duration of time followed by an interval of non-activation that is followed by a second activation period followed by a non-activation period and then by a third activation period, so on and so forth.

In certain embodiments, the temporal activation of the biological tissue 165 comprises activation of a first portion (not shown) of the biological tissue which is immediately followed by activation of a second portion (not shown) of biological tissue that is immediately followed activation of a third portion (not shown) of biological tissue, so on and so forth, until complete activation/coverage is achieved for all required portions of biological tissue and/or animal body.

A person of skill in the art will recognize that the selected dose corresponding to a predicted therapeutic level of electromagnetic energy for activation of different portions of a biological tissue may be different depending on palliative or therapeutic or other purposeful desired result to be achieved. Likewise, the selected dose corresponding to a predicted therapeutic level of electromagnetic energy required for partial and/or complete activation of a given biological tissue from a certain origin (animal or plant or microbial) may be different compared to the selected dose required for activation of a biological tissue of a different origin. In a similar vein, one of skill in the art will realize that the selected dose corresponding to a predicted therapeutic level of electromagnetic energy required for partial or complete activation of biological tissues from different parts of the same animal (or plant or microbial cells) may require a different the selected dose for obtaining a desired level of activation. For example, treatment regimes for brain tumors may differ based on the nature, origin, location (e.g. depth of location), shape and size of the tumors.

In an embodiment, an electromagnetic energy-activated biological tissue may include an electromagnetic energy-mediated activated biological tissue. The electromagnetic energy-mediated activated biological tissue may be activated to a state of a necrosis, an induced apoptosis, a non-lethal metabolic physiological alteration, and/or an enhancement of tissue function.

In some embodiments, the term “activation” includes achieving a desired purpose with electromagnetic energy at an anatomical site or area of a living body and/or biological tissue that is intended to receive the radiation as prescribed in a dosage regime.

An alternative embodiment may include a combination of an electromagnetic energy, a biological tissue, and an endogenous or an exogenous pharmacological agent or drug. In combination, the pharmacological agent or drug may be activated in vivo to achieve a conformational or a functional alteration with respect to the biological tissue. One skilled in the art will recognize that the combination of the electromagnetic energy, the biological tissue, and the agent or drug may be used to achieve a focalized activation state of a locally-distributed or a systemically-distributed agent or drug.

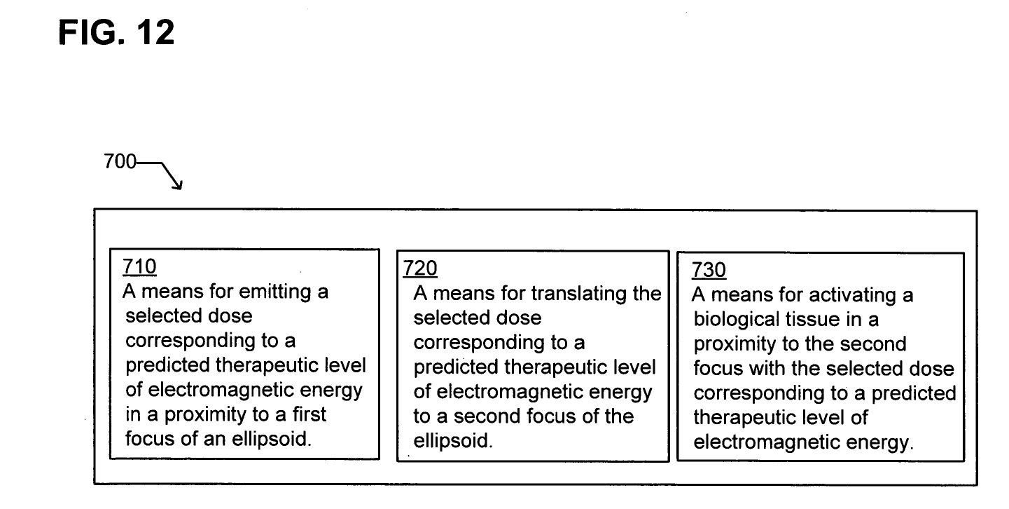

FIG. 12 illustrates an exemplary medical device 700 that may be used to implement embodiments. The device includes a means 710 for emitting a selected dose corresponding to a predicted therapeutic level of electromagnetic energy in a proximity to a first focus of an ellipsoid. The medical device also includes a means for translating 720 the selected dose corresponding to a predicted therapeutic level of electromagnetic energy to a second focus of the ellipsoid. The medical device further includes a means for activating 730 a biological tissue in a proximity to the second focus with the selected dose corresponding to a predicted therapeutic level of electromagnetic energy.

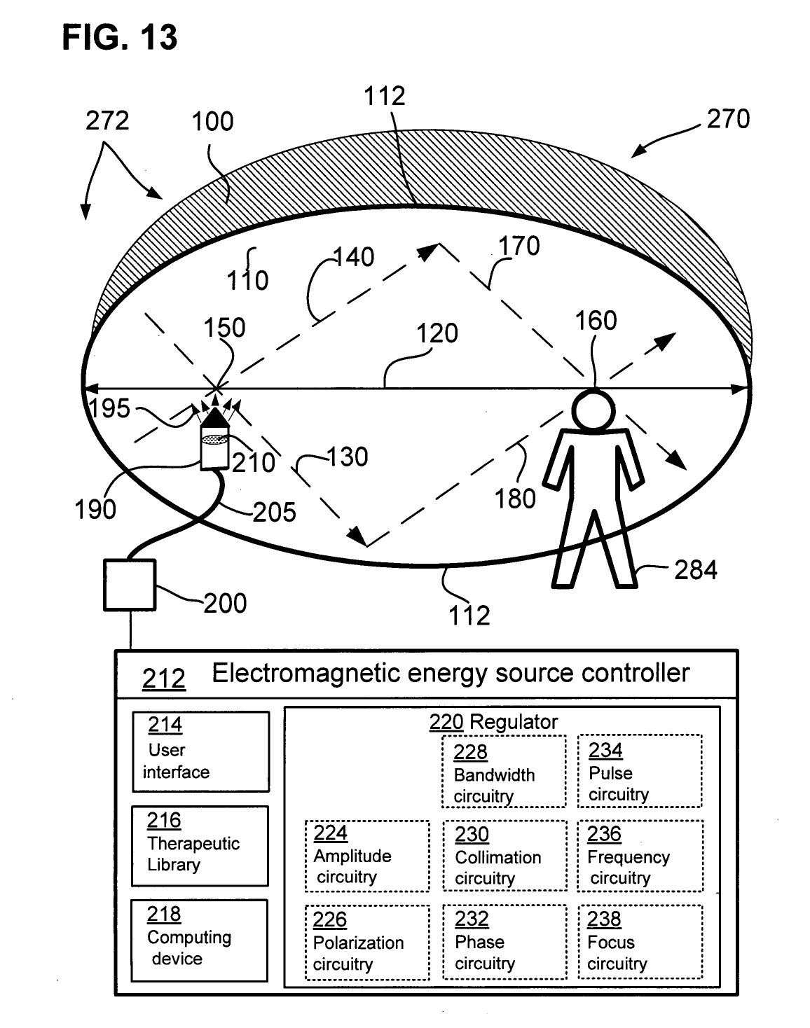

FIG. 13 illustrates an exemplary system 272 that may be used to implement embodiments. An embodiment of the system includes an ellipsoidally shaped reflector 100 having a first focus 150 and a second focus 160, shaped to provide a translational coupling 120 of non-biological electromagnetic energy 195 from the first focus to the second focus. A further embodiment of the system includes a controllable electromagnetic energy source 200 aligned to emit a non-biologically emitted electromagnetic energy in a proximity to the first focus. In yet another embodiment, the system includes an electromagnetic energy source controller 212 that is coupled to the energy source and having a regulator 220. In an embodiment, the electromagnetic energy source controller includes a user interface 214. In a further embodiment, the electromagnetic energy source controller includes a therapeutic library 216 that includes at least one of a treatment regime. In another embodiment, the electromagnetic energy source controller includes a computing device 218 that includes at least one computer. In a further embodiment, the regulator includes an amplitude-regulating electrical circuitry 224 configured to govern at least one amplitude emitted by the electromagnetic energy source. In another embodiment, the regulator includes a polarization-regulating electrical circuitry 226 configured to govern at least one polarization state emitted by the electromagnetic energy source. In yet another embodiment, the regulator includes a bandwidth-regulating electrical circuitry 228 configured to govern at least one bandwidth emitted by the electromagnetic energy source. In a further embodiment, the regulator includes a collimation-regulating electrical circuitry 230 configured to govern at least one collimation filter of the electromagnetic energy source. In an embodiment, the regulator includes a phase-regulating electrical circuitry 232 configured to govern at least one phase shift emitted by the electromagnetic energy source. In another embodiment, the regulator includes a pulse-regulating electrical circuitry 234 configured to govern at least one pulse emitted by the electromagnetic energy source. In a further embodiment, the regulator includes a frequency-regulating electrical circuitry 236 configured to govern at least one frequency emitted by the electromagnetic energy source. In an embodiment, the regulator includes a focus-regulating electrical circuitry 238 configured to govern at least one focal area of the electromagnetic energy.

Embodiments may be adapted for use in scanning and imaging devices working in conjunction with charge coupled devices. For instance, the non-biological electromagnetic energy source may be tuned to emit a specific bandwidth or wavelength of radiation to scan biological tissues, vascular structures, brain and/or other internal organs for scanning-imaging purposes. Embodiments may also be used in conjunction with fluorescence spectroscopy and/or diffuse reflectance spectroscopy/scattering technologies and/or optical spectroscopy and/or magnetic resonance spectroscopy of biological tissues. Likewise, embodiments may be adapted for skin exfoliation, skin rejuvenation treatments in conjunction with appropriate chemicals, for photodynamic therapy, collagen regenerative therapy, clearing blemishes, ex vivo blood purification therapy and ex situ imaging design. Another potential application is the adaptation of embodiments disclosed herein to imaging of tumors, biological tissue and/or whole bodies. Thus the embodiments may be adapted to operate in detection mode, demarcation mode, scanning mode and/or treatment mode.

The foregoing detailed description has set forth various embodiments of the devices and/or processes via the use of flowcharts, diagrams, figures and/or examples. Insofar as such flowcharts, diagrams, figures and/or examples contain one or more functions and/or operations, it will be understood by those within the art that each function and/or operation within such flowchart, diagram, figure and/or example can be implemented, individually and/or collectively, by a wide range of any combination thereof.

One skilled in the art will recognize that the herein described components (e.g., steps), devices, and objects and the discussion accompanying them are used as examples for the sake of conceptual clarity and that various configuration modifications are within the skill of those in the art. Consequently, as used herein, the specific exemplars set forth and the accompanying discussion are intended to be representative of their more general classes. In general, use of any specific exemplar herein is also intended to be representative of its class, and the non-inclusion of such specific components (e.g., steps), devices, and objects herein should not be taken as indicating that limitation is desired.

The herein described subject matter sometimes illustrates different components contained within, or connected with, different other components. It is to be understood that such depicted figures are merely exemplary, and that in fact many other figures can be implemented which achieve the same functionality. In a conceptual sense, any arrangement of components to achieve the same functionality is effectively “associated” such that the desired functionality is achieved. Hence, any two components herein combined to achieve a particular functionality can be seen as “associated with” each other such that the desired functionality is achieved, irrespective of architectures or intermedial components. Likewise, any two components so associated can also be viewed as being “operably connected”, or “operably coupled”, to each other to achieve the desired functionality, and any two components capable of being so associated can also be viewed as being “operably couplable”, to each other to achieve the desired functionality. Specific examples of operably couplable include but are not limited to physically mateable and/or physically interacting components and/or wirelessly interactable and/or wirelessly interacting components and/or logically interacting and/or logically interactable components.

In a general sense, those skilled in the art will recognize that the various aspects described herein which can be implemented, individually and/or collectively, by a wide range of hardware, software, firmware, or any combination thereof can be viewed as being composed of various types of “electrical circuitry.” Consequently, as used herein “electrical circuitry” includes, but is not limited to, electrical circuitry having at least one discrete electrical circuit, electrical circuitry having at least one integrated circuit, electrical circuitry having at least one application specific integrated circuit, electrical circuitry forming a general purpose computing device configured by a computer program (e.g., a general purpose computer configured by a computer program which at least partially carries out processes and/or devices described herein, or a microprocessor configured by a computer program which at least partially carries out processes and/or devices described herein), electrical circuitry forming a memory device (e.g., forms of random access memory), and/or electrical circuitry forming a communications device (e.g., a modem, communications switch, or optical-electrical equipment). Those having skill in the art will recognize that the subject matter described herein may be implemented in an analog or digital fashion or some combination thereof.

Those skilled in the art will recognize that it is common within the art to describe devices and/or systems in the fashion set forth herein, and thereafter use engineering practices to integrate such described devices and/or systems into electromagnetic radiation systems. That is, at least a portion of the devices and/or system described herein can be integrated into an electromagnetic radiation system via a reasonable amount of experimentation. Those having skill in the art will recognize that a typical electromagnetic radiation system generally includes one or more of a system unit housing, video display devices, memory such as volatile and non-volatile memory, processors such as microprocessors and digital signal processors, computational entities such as operating systems, drivers, and applications programs, one or more interaction devices, such as a touch pad or screen, control systems including feedback loops and control motors (e.g., feedback for sensing lens position and/or velocity; control motors for moving/distorting various optical and non-optical components. One skilled in the art will recognize that a typical electromagnetic radiation system includes, but is not limited to, a variety of optical and non-optical components such as lenses, filters, focusers, mirrors, collimators, monochromators, optical beam splitters, optical beam shifters, polarizers; wavelength, frequency, bandwidth, and/or phase modulators and/or controllers; optical and/or non-optical radiation emitters such as pulse and/or continuous lasers, arcs, lamps, LEDs, linear and/or nonlinear optical devices, radioactive element-based sources, micro wave emitters, ultra sonic and/or sonic emitters. A typical electromagnetic radiation system and/or an improvement thereof may be implemented utilizing one or more suitable commercially available components, including but not limited to the above-listed components.

While particular aspects of the present subject matter described herein have been shown and described, it will be apparent to those skilled in the art that, based upon the embodiments herein, changes and modifications may be made without departing from the subject matter described herein and its broader aspects and, therefore, the appended claims are to encompass within their scope all such changes and modifications as are within the true spirit and scope of the subject matter described herein. Furthermore, it is to be understood that the invention is defined by the appended claims. It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to inventions containing only one such recitation, even when the same claim includes the introductory phrases “one or more” or “at least one” and indefinite articles such as “a” or “an” (e.g., “a” and/or “an” should typically be interpreted to mean “at least one” or “one or more”); the same holds true for the use of definite articles used to introduce claim recitations. In addition, even if a specific number of an introduced claim recitation is explicitly recited, those skilled in the art will recognize that such recitation should typically be interpreted to mean at least the recited number (e.g., the bare recitation of “two recitations,” without other modifiers, typically means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

1. A medical device, comprising:
an ellipsoidally shaped reflector having a first focus and a second focus, and providing a translational coupling of electromagnetic energy from said first focus to said second focus; and
a controllable electromagnetic energy source aligned to emit a non-biologically emitted electromagnetic energy in a proximity to said first focus.

2. The device of claim 1, wherein said ellipsoidally shaped reflector includes an opening configured to allow positioning of a portion of a body proximate with said second focus.

3. The device of claim 1, wherein said ellipsoidally shaped reflector includes a conductor.

4. The device of claim 3, wherein said conductor includes at least one of an aluminum, a tin, a stainless steel, a silver, a gold, a copper, an iron, a carbon, an iridium, an indium, a lead, a magnesium, a nickel, a nichrome, a palladium, a rhodium, a silver, a tantalum, a titanium, a tungsten, a zinc, a platinum and/or a zirconium.

5. The device of claim 1, wherein said ellipsoidally shaped reflector includes a dielectric material.

6. The device of claim 5, wherein said dielectric material includes a rubber, a plastic, a porcelain, a ceramic, a mica, a glass, a metal oxide, a perfect vacuum, a dry air, a pure dry gas, a helium and/or a nitrogen.

7. The device of claim 1, wherein said non-biologically emitted electromagnetic energy includes at least one of a visible light, a laser energy, an ultraviolet energy, an infrared energy, an X-ray, and/or a microwave.

8. The device of claim 1, wherein said controllable electromagnetic energy source includes a computer configured to control delivery of said non-biological electromagnetic energy.

9. The device of claim 1, wherein said controllable electromagnetic energy source includes at least one of a plano-convex lens, a meniscus lens, a cylindrical lens, a parabolic lens, an acrylic lens, a glass lens, a quartz lens, a Fresnel lens, a Pendry lens, a band pass filter, a polarizer, a dichroic material, a monochromator and/or a collimator.

10. The device of claim 1, wherein said controllable electromagnetic energy source regulates at least a characteristic of said non-biologically emitted electromagnetic energy.

11. The device of claim 10, wherein said at least a characteristic of said non-biologically emitted electromagnetic energy includes at least one of a wavelength, a frequency, an amplitude, a phase, a polarization and/or a bandwidth.

12. A method of treating a portion of a living body, comprising:
emitting a selected dose corresponding to a predicted therapeutic level of electromagnetic energy in a proximity to a first focus of an ellipsoid;
translating said selected dose corresponding to a predicted therapeutic level of electromagnetic energy to a second focus of said ellipsoid; and
activating a biological tissue in a proximity to said second focus with said selected dose corresponding to a predicted therapeutic level of electromagnetic energy.

13. The method of claim 12, wherein said emitting a selected dose corresponding to a predicted therapeutic level of electromagnetic energy in a proximity to a first focus of an ellipsoid includes emitting said selected dose corresponding to a predicted therapeutic level of electromagnetic energy having at least one wavelength between 1 nm and 700 nm, 2 μm and 10 μm and/or 1 cm and 100 cm.

14. The method of claim 12, wherein said emitting a selected dose corresponding to a predicted therapeutic level of electromagnetic energy in a proximity to a first focus of an ellipsoid includes emitting said selected dose corresponding to a predicted therapeutic level of electromagnetic energy having at least one wavelength between 10 μm and 1 cm.

15. The method of claim 12, wherein said emitting a selected dose corresponding to a predicted therapeutic level of electromagnetic energy in a proximity to a first focus of an ellipsoid includes emitting said selected dose corresponding to a predicted therapeutic level of electromagnetic energy having at least one wavelength between 700 and 2000 nm.

16. The method of claim 12, wherein said emitting a selected dose corresponding to a predicted therapeutic level of electromagnetic energy in a proximity to a first focus of an ellipsoid includes emitting said selected dose corresponding to a predicted therapeutic level of electromagnetic energy having at least one of a wavelength, an amplitude variation, a phase variation and/or a variable polarization parameter.

17. The method of claim 12, wherein said emitting a selected dose corresponding to a predicted therapeutic level of electromagnetic energy in a proximity to a first focus of an ellipsoid includes emitting said selected dose corresponding to a predicted therapeutic level of electromagnetic energy as a series of one or more pulses, each of said pulses having at least a pulse duration between a picosecond and a second.

18. The method of claim 12, wherein translating said selected dose corresponding to a predicted therapeutic level of electromagnetic energy to a second focus of said ellipsoid includes said second focus having a volume between 1000 μm³ and 1000 cm³ in a proximity to a biological tissue.

19. The method of claim 12, wherein said activating a biological tissue in a proximity to said second focus with said selected dose corresponding to a predicted therapeutic level of electromagnetic energy includes selectively energizing a first portion of the biological tissue differentially relative to a second portion of the biological tissue.

20. The method of claim 12, wherein said activating a biological tissue in a proximity to said second focus with said selected dose corresponding to a predicted therapeutic level of electromagnetic energy includes coverage of 0.1% to 100% of said biological tissue by said second focus.

21. The method of claim 12, wherein said activating a biological tissue in a proximity to said second focus with said selected dose corresponding to a predicted therapeutic level of electromagnetic energy includes activating said biological tissue using electromagnetic energy having a level between 1 to 100,000 milli Joules per gram of biological tissue at said second focus.

22. The method of claim 12, wherein said activating a biological tissue in a proximity to said second focus with said selected dose corresponding to a predicted therapeutic level of electromagnetic energy includes making said second focus at least substantially coincidental with a first portion of said biological tissue and then making said second focus at least substantially coincidental with a second portion of said biological tissue.

23. A medical device, comprising:
means for emitting a selected dose corresponding to a predicted therapeutic level of electromagnetic energy in a proximity to a first focus of an ellipsoid;
means for translating said selected dose corresponding to a predicted therapeutic level of electromagnetic energy to a second focus of said ellipsoid; and
means for activating a biological tissue in a proximity to said second focus with said selected dose corresponding to a predicted therapeutic level of electromagnetic energy.

24. A system comprising:
an ellipsoidally shaped reflector having a first focus and a second focus, shaped to provide a translational coupling of electromagnetic energy from said first focus to said second focus;
a controllable electromagnetic energy source aligned to emit a non-biologically emitted electromagnetic energy in a proximity to said first focus; and
an electromagnetic energy source controller coupled to said energy source and having a regulator.

25. The system of claim 24, having an electromagnetic energy source controller coupled to said energy source that includes a user interface.

26. The system of claim 24, having an electromagnetic energy source controller coupled to said energy source that includes a therapeutic library having at least one of a treatment regime.

27. The system of claim 24, having an electromagnetic energy source controller coupled to said energy source having a computing device that includes at least one computer.

28. The system of claim 24, wherein said regulator includes electrical circuitry configured to govern at least one wavelength emitted by said electromagnetic energy source.

29. The system of claim 24, wherein said regulator includes electrical circuitry configured to govern at least one amplitude emitted by said electromagnetic energy source.

30. The system of claim 24, wherein said regulator includes electrical circuitry configured to govern at least one polarization state emitted by said electromagnetic energy source.

31. The system of claim 24, wherein said regulator includes electrical circuitry configured to govern at least one bandwidth emitted by said electromagnetic energy source.

32. The system of claim 24, wherein said regulator includes electrical circuitry configured to govern at least one collimation filter of said electromagnetic energy source.

33. The system of claim 24, wherein said regulator includes electrical circuitry configured to govern at least one pulse emitted by said electromagnetic energy source.

34. The system of claim 24, wherein said regulator includes electrical circuitry configured to govern at least one phase shift emitted by said electromagnetic energy source.

35. The system of claim 24, wherein said regulator includes electrical circuitry configured to govern at least one focal area of said electromagnetic energy.
